(19) United States
(12) Patent Application Publication
(10) Pub. No.: US 2019/0167260 A1
(43) Pub. Date: Jun. 6, 2019

(54) Fixation Device

(71) Applicant: Duke University, Durham, NC (US)

(72) Inventors: Howard Levinson, Durham, NC (US); Kenneth A. Gall, Durham, NC (US)

(21) Appl. No.: 16/323,656

(22) PCT Filed: Aug. 8, 2017
(86) PCT No.: PCT/US17/45829

§ 371 Date: Feb. 6, 2019

Related U.S. Application Data
(60) Provisional application No. 62/371,907, filed on Aug. 8, 2016.

(51) Int. Cl. A61B 17/04; A61L 31/04; A61L 31/14

(52) U.S. Cl. CPC A61B 17/0487; A61B 2017/00477; A61L 31/14; A61L 31/041

(57) ABSTRACT

A fixation device is disclosed. The fixation device includes a first component and a second component. The first component has a top surface, a base, at least one locking projection, and at least one tapered projection extending from the top surface. The at least one locking projection includes a first locking element. The fixation device further includes a second component having a top surface, a base, at least one locking aperture, and at least one second aperture corresponding to the projections on the first component. The at least one locking apertures includes a second locking element. In some embodiments, the at least one locking projection on the first component is configured to lock together with the at least one locking aperture on the second component to secure together two ends of a surgical mesh suture or tape suture.

### CROSS-REFERENCE TO RELATED APPLICATIONS

This application claims priority to U.S. Provisional Application No. 62/371,907 filed Aug. 8, 2016, which is hereby incorporated by reference in its entirety.

### BACKGROUND

### A. Field

This disclosure relates generally to fixation devices and more particularly to a fixation device or anchor for fastening two ends of a surgical suture.

### B. Description of Related Art

Wide sutures are used to close high-tension wounds such as in hernia or tendon repair, and may be used for soft-tissue to soft tissue fixation, soft tissue to bone fixation, or bone to bone fixation. Two types of wide sutures are tape sutures and mesh sutures. Despite the many advantages of wide sutures, a concern in using these sutures are the size of the large knots needed to anchor or secure the two ends of the suture. Large knots are susceptible to increased palpability, pain, foreign body response, and may increase the risk of infection. Thus, there is a need for an alternative suture fixation or suture anchor device to replace large knots when securing mesh sutures or tape sutures within a patient.

### SUMMARY

The following embodiments and aspects thereof are described and illustrated in conjunction with systems, tools and methods which are meant to be exemplary and illustrative, not limiting in scope.

In one aspect, a fixation device is disclosed. The fixation device may include a first component and a second component. The first component has a top surface, a base, at least one locking projection, and at least one tapered projection extending from the top surface. The at least one locking projection includes a first locking element. The fixation device further includes a second component having a top surface, a base, at least one locking aperture, and at least one second aperture corresponding to the projections on the first component. The at least one locking apertures includes a second locking element. In some embodiments, the at least one locking projection on the first component is configured to lock together with the at least one locking aperture on the second component to secure together two ends of a mesh suture or a tape suture.

In another aspect, the fixation device includes a male component having a top surface, a base, two locking projections, and a tapered projection extending from the top surface. The two locking projections each include a plurality of first locking elements. The fixation device further includes a female component having a top surface, a base, two locking apertures, and a third aperture corresponding to the projections on the male component. The two locking apertures each include a plurality of second locking elements. The two locking projections on the male component are configured to lock together with the two locking apertures on the female component to secure together two ends of a mesh suture or tape suture.

In yet another embodiment, a fixation device includes a first component having a top surface, a base, and a plurality of first projections extending from the top surface. The plurality of first projections may be formed as first tines. The fixation device further includes a second component having a top surface, a base, and a plurality of second projections extending from the top surface configured to interlock with the plurality of first projections on the first component to secure together two ends of a mesh suture or tape suture. The fixation device also includes a hinge connecting the first component to the second component.

In addition to the exemplary aspects and embodiments described above, further aspects and embodiments will become apparent by reference to the drawings and by study of the following detailed description.

### BRIEF DESCRIPTION OF THE DRAWINGS

Exemplary embodiments are illustrated in the drawings. It is intended that the embodiments and figures disclosed herein are to be considered illustrative rather than restrictive.

FIG. 1 is a perspective view of a fixation device of the present application;

FIG. 2 is a front view of the fixation device shown in FIG. 1;

FIG. 3 shows the fixation device of FIG. 1 in a secured position;

FIG. 4A shows an alternate embodiment of a fixation device of the present application;

FIG. 4B shows the fixation device of FIG. 4A in a secured position; and

FIG. 4C shows a side view of the fixation device of FIG. 4B.

### DETAILED DESCRIPTION

A fixation device for fastening two ends of a surgical mesh suture or tape suture is disclosed. The fixation device may include a first component and a second component which interlock with each other. In some embodiments, the first component may be a male component having one or more projections and the second component may be a female component having one or more apertures or recesses. At least one of the projections may have one or more projection locking elements, and at least one of the apertures may have one or more corresponding aperture locking elements. Moreover, the fixation device may be made of a biocompatible material, such as silicone, PEEK, or any other biocompatible material, for example.

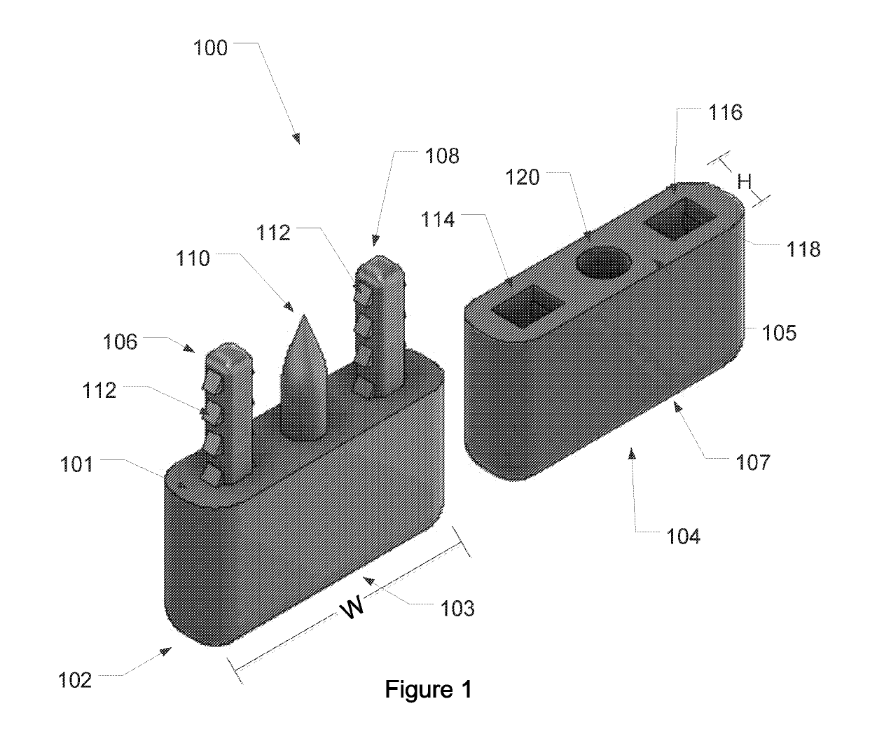

FIG. 1 shows an example fixation device 100 of the present application. The fixation device 100 may include two interlocking components: a first, male component 102 and a second, female component 104. The male component 102 may have a substantially flat top surface 101 and a base portion 103. The male component 102 may include a plurality of projections extending from the top surface 101. In one embodiment, the male component 102 includes two lateral locking projections 106, 108 and a middle projection 110 located between the lateral locking projections 106, 108. Each of the projections extends outwardly from the top surface 101.

The lateral locking projections 106, 108 each include one or more locking elements 112 to enable locking when attached to the female component 104. In one embodiment, the projection locking elements 112 comprise a plurality of pointed, barbed-shaped projections. However, it should be understood that any suitable locking elements may be used.

The middle projection 110 may be tapered distally to enable penetration through a suture. The middle projection 110 provides suture fixation via a cotter pin-like mechanism. Other shapes and structures of the middle projection 110 are possible as well.

Referring again to FIG. 1, the female component 104 may include corresponding apertures or holes to enable entry and locking of the projections 106, 108, and middle projection 110. The female component 104 may include a top surface 105 and a base portion 107. In one embodiment, locking apertures 114, 116 are formed in the top surface 105 to correspond to the lateral locking projections 106, 108. The locking apertures 114, 116 each include one or more locking elements 118 to enable locking when attached to the male component 102. In one example, the aperture locking elements 118 comprise a plurality of notches which mate with the locking elements 112 of lateral locking projections 106, 108 to lock the male component 102 to the female component 104. However, in other embodiments, any suitable locking elements may be used.

A middle aperture 120 may be located on the female component 104 between the two locking apertures 114, 116. The middle aperture 120 may be shaped and sized to accommodate the middle projection 110 of male component 102.

It should be understood that in alternate embodiments the first component may be the female component having apertures and the second component may be the male component having projections. Additionally, in some embodiments, the first component may include a mix of projections and apertures and the second component may include a corresponding mix of apertures and projections.

Moreover, additional or less projections can be formed on the male component 102, with each having a corresponding aperture formed on the female component 104. Additionally, although the male component 102 and the female component 104 both have a generally rectangular shape, in alternate embodiments, other suitable shapes are also possible.

In one embodiment, the height H of the fixation device 100, shown in FIG. 1, may be about 2 mm. The width W and length L of the assembled fixation device 100 (shown in FIGS. 1-3) may each be about 10 mm. In another embodiment, the height H of the fixation device may be in the range of 0.1 cm to 1 cm, the width W may be in the range of 0.5 cm to 2 cm, and the length L may be in the range of 0.2 cm to 2 cm. Other suitable dimensions are also possible.

The fixation device 100 may comprise a biocompatible material, such as silicone, for example. In alternate embodiments, the fixation device 100 may comprise any other suitable biocompatible materials as well, including, but not limited to silicone, Polyether ether ketone (PEEK), Polyethylene, polylactide (PLA), stainless steel, titanium, cobalt chrome, or other biocompatible polymer or metallic alloy or combinations thereof.

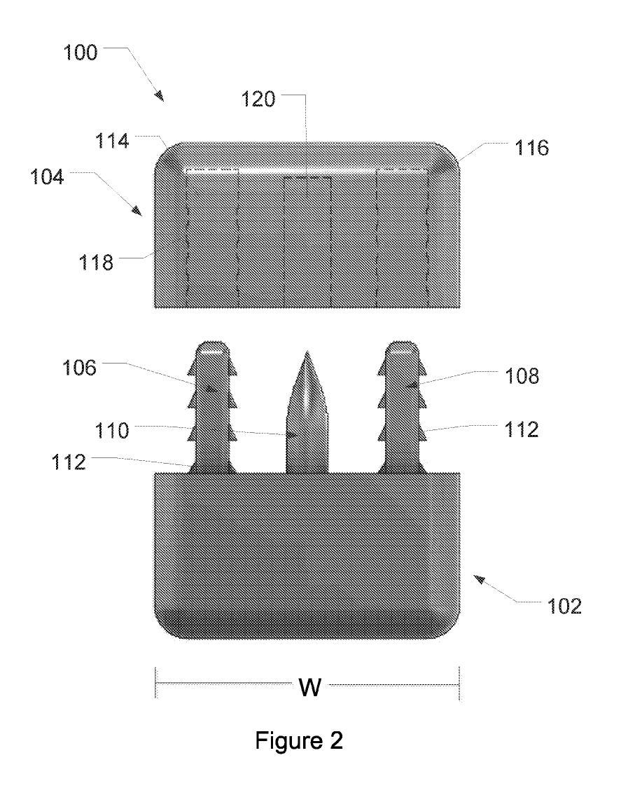

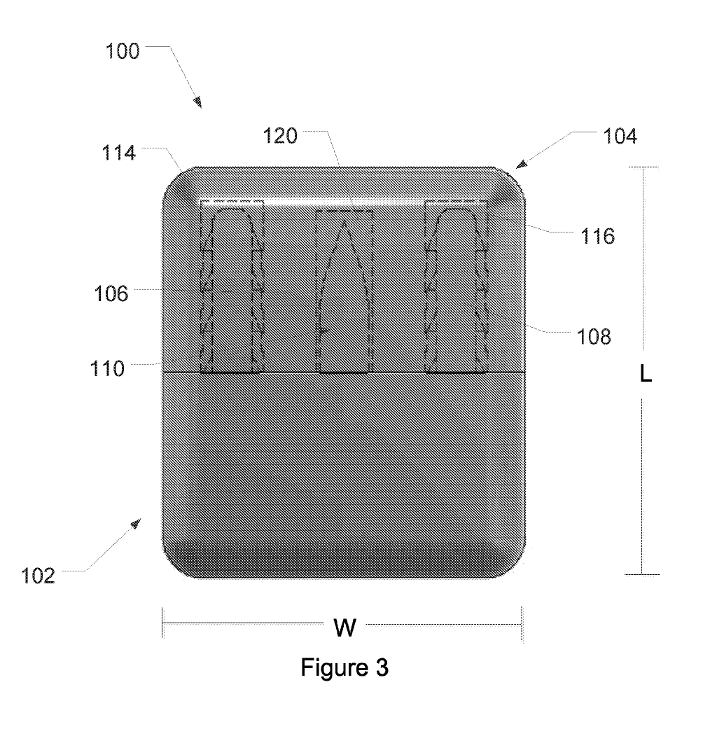

Referring now to FIGS. 2 and 3, the male and female components 102, 104 of the fixation device 100 can be assembled and secured together by inserting the projections 106, 108, 110 into the apertures 114, 116, 120. In one embodiment, the male and female components 102, 104 of the fixation device 100 are placed around a portion of a surgical mesh (not shown) to secure the mesh within a patient. The male and female components 102, 104 may be clamped or closed together around the surgical mesh with a handheld or laparoscopic clip applier (not shown). Once secured together, the locking elements 112 on the locking projections 106, 108 engage with the locking elements 118 on the locking apertures 114, 116.

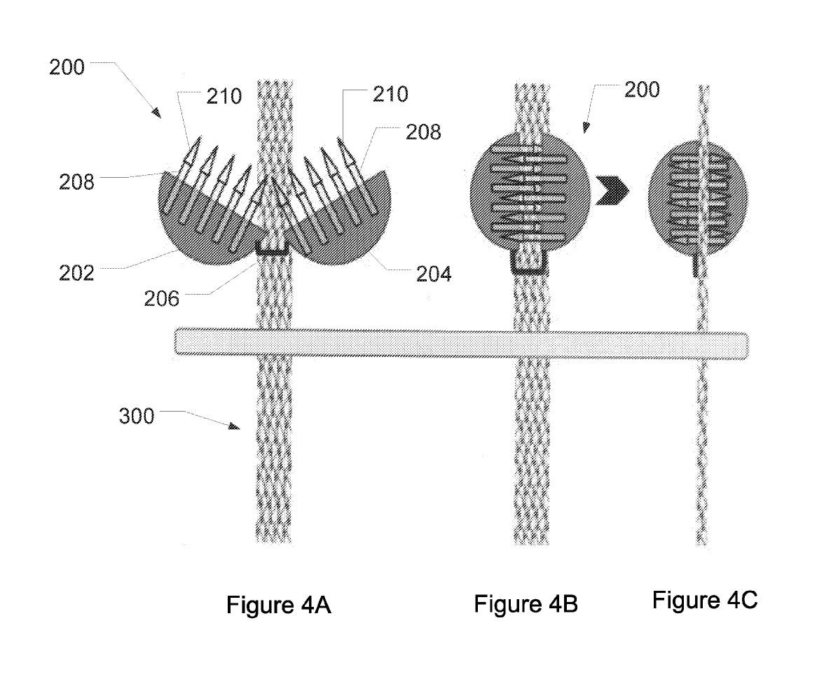

FIGS. 4A-4C show an alternate embodiment of a fixation device 200 of the present application. The fixation device 200 may comprise a button-type structure which secures a mesh extension for a T-line hernia mesh 300. In one embodiment, the fixation device 200 comprises a first component 202 and second component 204. The first and second components 202, 204 may be formed as discs. In some embodiments, the discs are semi-circular in shape. It should be understood that the first and second components 202, 204 may take other suitable shapes as well. In some embodiments, first component 202 may be a different shape than second component 204.

The first and second components 202, 204 may be connected by a hinge piece 206. Thus, the first and second components 202, 204 may easily be moved together and apart. The hinge piece 206 may be made of any suitable material. It should be understood that any suitable hinge may be used.

The first and second components 202, 204 may each include a plurality of projections 208. In one embodiment, the projections 208 are formed as tines. In some embodiments, the tines include a bishop-shaped or tapered head 210. When the first and second components 202, 204 are secured together, the tines 208 interlock to form a secure connection.

In some embodiments, the first and second components 202, 204 comprise a biocompatible material, such as silicone. In alternate embodiments, the fixation device 200 may comprise any other suitable biocompatible materials as well.

As shown in FIGS. 4A-4C, the fixation device 200 is placed around a portion of a surgical mesh suture or tape suture 300 to secure the mesh within a patient. In some embodiments, the first and second components 202, 204 may be clamped/closed together around the mesh extension 300 with a handheld or laparoscopic clip applier (not shown).

While a number of exemplary aspects and embodiments have been discussed above, those of skill in the art will recognize that still further modifications, permutations, additions and sub-combinations thereof of the features of the disclosed embodiments are still possible. It is therefore intended that the following appended claims and claims hereafter introduced are interpreted to include all such modifications, permutations, additions and sub-combinations as are within their true spirit and scope.

What is claimed is:

1. A fixation device comprising:
a first component having a top surface and a base, the first component including at least one locking projection and at least one tapered projection extending from the top surface, wherein the at least one locking projection includes a first projection locking element; and
a second component having a top surface and a base, the second component including at least one locking aperture and at least one second aperture corresponding to the projections on the first component, wherein the at least one locking apertures includes a first aperture locking element;
wherein the at least one locking projection on the first component is configured to lock together with the at least one locking aperture on the second component to secure together two ends of a mesh suture or tape suture.

2. The fixation device of claim 1 wherein the first component further comprises a second locking projection having a second projection locking element.

3. The fixation device of claim 2 wherein the second component comprises a second locking aperture having a second aperture locking element.

4. The fixation device of claim 3 wherein the first and second projection locking elements comprise barbs and the first and second aperture locking elements comprise notches.

5. The fixation device of claim 2 wherein the at least one tapered projection is positioned in between the first locking projection and the second locking projection.

6. The fixation device of claim 1 wherein the first component and second component comprise a biocompatible material.

7. The fixation device of claim 6 wherein the biocompatible material comprises any of silicone, PEEK, Polyethylene, PLA, Stainless steel, Titanium, cobalt chrome, or other biocompatible polymer or metallic alloy or combinations thereof.

8. A fixation device comprising:
a male component having a top surface and a base, the male component including two locking projections and a tapered projection extending from the top surface, wherein the two locking projections each include a plurality of first locking elements; and
a female component having a top surface and a base, the female component including two locking apertures and a third aperture corresponding to the projections on the male component, wherein the two locking apertures each include a plurality of second locking elements;
wherein the two locking projections on the male component are configured to lock together with the two locking apertures on the female component to secure together two ends of a mesh suture or tape suture.

9. The fixation device of claim 8 wherein the first locking elements comprise barbs and the second locking elements comprise notches.

10. The fixation device of claim 8 wherein the tapered projection is positioned in between the two locking projections.

11. The fixation device of claim 8 wherein the first component and second component comprise a biocompatible material.

12. The fixation device of claim 11 wherein the biocompatible material comprises any of silicone, PEEK, Polyethylene, PLA, Stainless steel, Titanium, cobalt chrome, or other biocompatible polymer or metallic alloy or combinations thereof.

13. A fixation device comprising:
a first component having a top surface and a base, the first component including a plurality of first projections extending from the top surface, wherein the plurality of first projections include first tines;
a second component having a top surface and a base, the second component including a plurality of second projections extending from the top surface configured to interlock with the plurality of first projections on the first component to secure together two ends of a mesh suture or tape suture; and
a hinge connecting the first component to the second component.

14. The fixation device of claim 13 wherein the first component and the second component each comprises disc shape.

15. The fixation device of claim 14 wherein the discs are semi-circular in shape.

16. The fixation device of claim 13 wherein the plurality of second projections include second tines.

17. The fixation device of claim 16 wherein the first tines and the second tines each include a tapered head.

18. The fixation device of claim 13 wherein the first component and second component comprise a biocompatible material.

19. The fixation device of claim 18 wherein the biocompatible material comprises any of silicone, PEEK, Polyethylene, PLA, Stainless steel, Titanium, cobalt chrome, or other biocompatible polymer or metallic alloy or combinations thereof.
